(19) United States
(12) Patent Application Publication
(10) Pub. No.: US 2010/0010626 A1
(43) Pub. Date: Jan. 14, 2010

(54) ARTIFICIAL EYE

(75) Inventors: CHUN-YU LEE, Tu-Cheng (TW); TSUNG-YU LIN, Tu-Cheng (TW)

Correspondence Address: PCE INDUSTRY, INC.;ATT. Steven Reiss, 288 SOUTH MAYO AVENUE, CITY OF INDUSTRY, CA, 91789, US

(73) Assignee: HON HAI PRECISION INDUSTRY CO., LTD., Tu-Cheng (TW)

(21) Appl. No.: 12/399,208

(22) Filed: Mar. 6, 2009

(30) Foreign Application Priority Data
Jul. 8, 2008 (CN) 200810302642.1

(51) Int. Cl. A61F 2/14

(52) U.S. Cl. 623/6.64

(57) ABSTRACT

An artificial eye includes a supporter, a first clipping member, a second clipping member and an electroactive pupil. The second clipping member is positioned on the supporter. A peripheral portion of the second clipping member is positioned on a peripheral portion of the first clipping member. A gap is defined between the first and second clipping members. The electroactive pupil is positioned in the gap and contacts the first and second clipping members.

### BACKGROUND

1. Technical Field

The disclosure relates to an artificial eye.

2. Description of Related Art

In order to enhance playing enjoyment, various dolls or robots are provided to simulate human expressions, such as the closing and the opening of eyes. However, a typical artificial eye has a pupil that does not change with respect to different conditions. As a result, the typical artificial eye can not realistically simulate a human's eye.

Therefore, a new artificial eye is desired to overcome the above-described shortcoming.

### BRIEF DESCRIPTION OF THE DRAWINGS

Many aspects of the embodiments can be better understood with reference to the following drawings. The components in the drawings are not necessarily drawn to scale, the emphasis instead being placed upon clearly illustrating the principles of the embodiments. Moreover, in the drawings, like reference numerals designate corresponding parts throughout the several views.

FIG. 1 is a perspective view of one embodiment of an artificial eye.

FIG. 2 is a cross-sectional view of the artificial eye of FIG. 1, taken alone line II-II.

FIG. 3 is a cross-sectional view of a pupil assembly of the artificial eye of FIG. 1.

FIG. 4 is a cross-sectional view of the pupil assembly, a pupil of the pupil assembly expanding.

FIG. 5 is a perspective view of the artificial eye, the pupil expanding.

FIG. 6 is a cross-sectional view of one embodiment of a pupil assembly.

FIG. 7 is a cross-sectional view of another embodiment of an artificial eye.

### DETAILED DESCRIPTION OF THE EMBODIMENTS

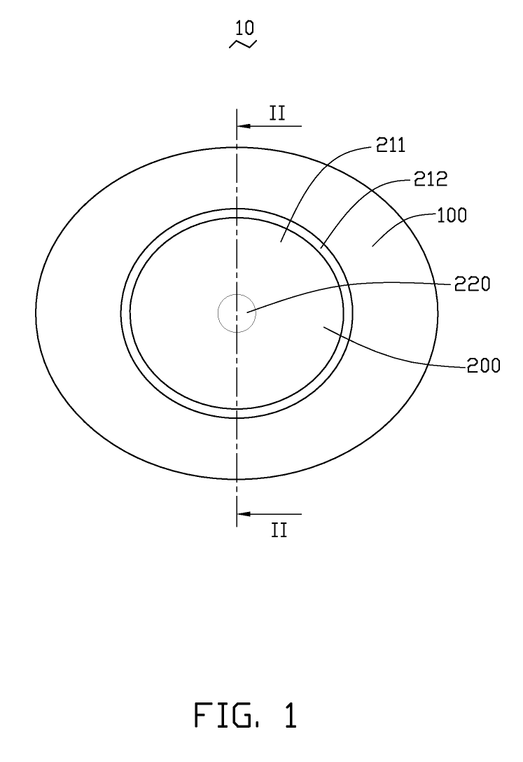

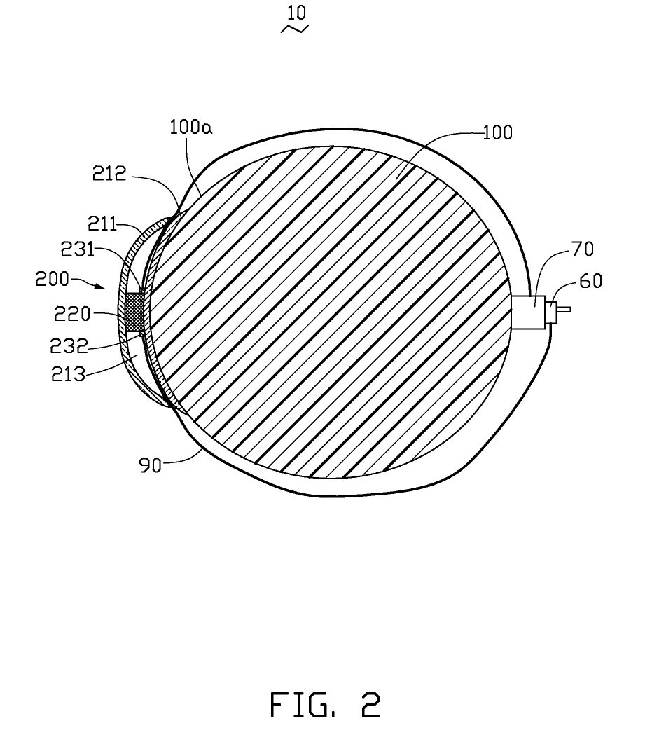

Referring to FIGS. 1 and 2, one embodiment of an artificial eye 10 includes a supporter 100 and a pupil assembly 200 positioned on the supporter 100.

The supporter 100 may be a substantially spherical-shaped body, and made of an elastic material. The supporter 100 has a surface 100a.

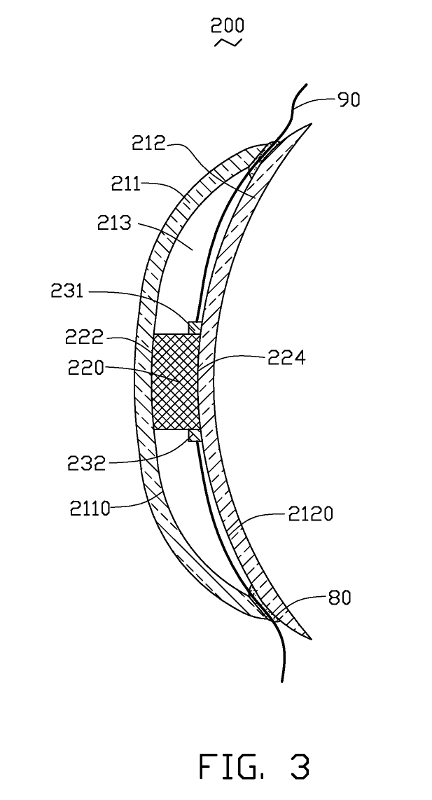

Also referring to FIG. 3, the pupil assembly 200 includes a first substantially circular-shaped clipping member 211, a second substantially circular-shaped clipping member 212, an electroactive pupil 220, a first electrode 231 and a second electrode 232. Each of the first and second clipping members 211, 212 may be a curved sheet. The first clipping member 211 has a concave surface 2110. The second clipping member 212 has a convex surface 2120. Peripheral portions of the first and second clipping members 211, 212 contact each other. In one embodiment, adhesive material members 80 are positioned between the peripheral portions to fix the first and second clipping members 211, 212 together. A gap 213 is defined between the concave surface 2110 and the convex surface 2120. The gap 213 may be a void space. The second clipping member 212 is positioned on the surface 100a. The first and second clipping members 211, 212 may be made of transparent materials, such as glass material, colored glass material, or polymethyl methacrylate (PMMA).

The electroactive pupil 220 is positioned in the gap 213 between the concave surface 2110 and the convex surface 2120, and made of an electroactive material, such as ion exchange membrane, gel polymer, electroactive polymer or piezoelectric polymer. The electroactive pupil 220 may be substantially cylindrical or spherical shaped. In the illustrated embodiment, the electroactive pupil 220 is substantially cylindrical shaped, has a first end 222 and a second end 224 opposite to the first end 222. The first end 222 contacts the concave surface 2110. The second end 224 contacts the convex surface 2120.

The first and second electrodes 231, 232 are positioned at two opposite ends of the electroactive pupil 220. The first electrode 231 is electrically coupled to a power source 70 via one conductive wire 90. The second electrode 232 is electrically coupled to a switch 60 via another conductive wire 90. The switch 60 is electrically coupled to the power source 70 and configured to turn on and turn off the power source 70. As a result, the electroactive pupil 220, the first and the second electrodes 231, 232, the power source 70 and the switch 60 constitute a circuit.

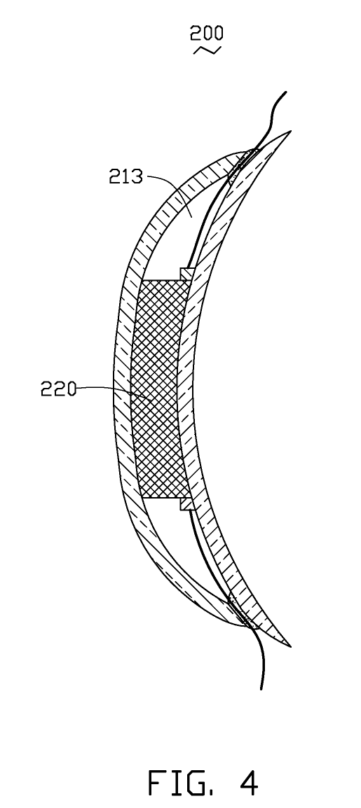

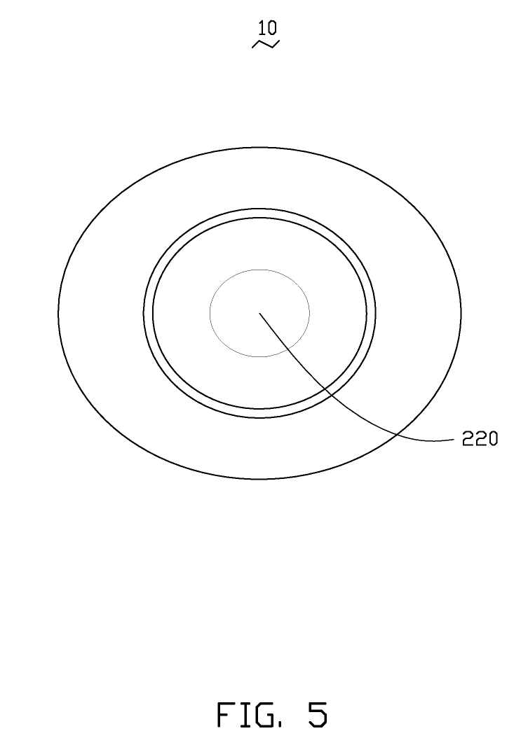

Also referring to FIGS. 4 and 5, in use, when the switch 60 is controlled to turn the power source 70 on, the electroactive pupil 220 is powered on to expand into the gap 213. When the switch 60 is controlled to turn the power source 70 off, the electroactive pupil 220 shrinks. Therefore, the artificial eye 10 can more realistically simulate a human eye.

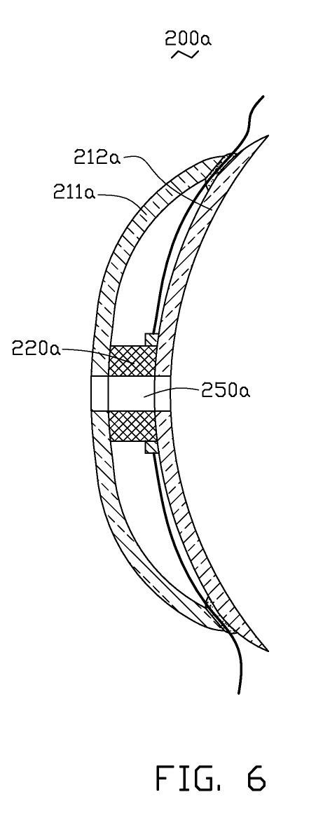

Referring to FIG. 6, another embodiment of a pupil assembly 200a is similar to the pupil assembly 200 of FIG. 3, except that a through hole 250a is defined in the first clipping member 211a, the pupil 220a and the second clipping member 212a. The through hole 250a allows a digital camera to be positioned therein.

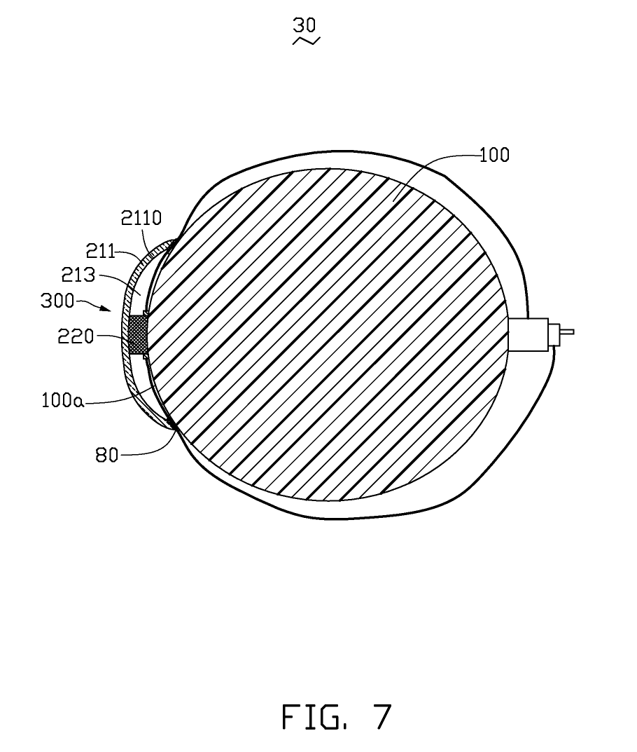

Referring to FIG. 7, another embodiment of an artificial eye 30 is similar to the artificial eye 10 of FIGS. 1 through 3, except that the electroactive pupil assembly 300 includes the clipping member 211 and the electroactive pupil 220. The gap 213 is defined between the concave surface 2110 of the clipping member 211 and the surface 100a. The electroactive pupil 220 is positioned between the concave surface 2110 and the surface 100a. The adhesive material members 80 are positioned between peripheral portion of the clipping member 211 and the surface 100a, thereby fixing the clipping member 211 to the supporter 100.

It is believed that the present embodiments and their advantages will be understood from the foregoing description, and it will be apparent that various changes may be made thereto without departing from the spirit and scope of the embodiments or sacrificing all of its material advantages.

What is claimed is:

1. An artificial eye, comprising:
a supporter;
a first clipping member;
a second clipping member positioned on the supporter, wherein a peripheral portion of the second clipping member is positioned on a peripheral portion of the first clipping member, and wherein a gap is defined between the first and second clipping members; and
an electroactive pupil positioned in the gap and contacting the first and second clipping members.

2. The artificial eye of claim 1, wherein the first and second clipping members are substantially circular shaped.

3. The artificial eye of claim 1, wherein each of the first and second clipping members is a curved sheet; the first clipping member has a concave surface; the second clipping member has a convex surface; the gap is defined between the concave surface and the convex surface; the electroactive pupil contacts the concave surface and the convex surface.

4. The artificial eye of claim 1, wherein a through hole is defined in the first clipping member, the electroactive pupil, and the second clipping member.

5. The artificial eye of claim 4, wherein a digital camera is positioned in the through hole.

6. The artificial eye of claim 1, wherein the electroactive pupil is made of an electroactive material.

7. The artificial eye of claim 1, further comprising a first electrode and a second electrode, wherein the first and second electrodes are positioned at two opposite ends of the electroactive pupil.

8. The artificial eye of claim 7, further comprising a power source electrically coupled to the first electrode, and a switch electrically coupled to the second electrode and the power source.

9. An artificial eye for a doll, comprising:
a supporter;
a clipping member, wherein a peripheral portion of the clipping member is positioned on the supporter, and wherein a gap is defined between the clipping member and the supporter; and
an electroactive pupil positioned in the gap and contacting the clipping member and the supporter.

10. The artificial eye of claim 9, wherein the clipping member is substantially circular shaped.

11. The artificial eye of claim 9, wherein the clipping member is a curved sheet and has a concave surface, wherein the gap is defined between the concave surface and the supporter.

12. The artificial eye of claim 9, wherein the clipping member is made of a transparent material.

13. The artificial eye of claim 9, wherein the electroactive pupil is made of an electroactive material.

14. The artificial eye of claim 9, wherein the electroactive pupil is substantially cylindrical or spherical shaped.

15. The artificial eye of claim 9, further comprising a first electrode and a second electrode, wherein the first and second electrodes are positioned at two opposite ends of the electroactive pupil.

16. The artificial eye of claim 15, further comprising a power source electrically coupled to the first electrode, and a switch electrically coupled to the second electrode and the power source.

17. A pupil assembly for an artificial eye, comprising:
a first curved sheet substantially circular shaped and having a concave surface; and
an electroactive pupil positioned on the concave surface.

18. The pupil assembly of claim 17, further comprising a second curved sheet substantially circular shaped and having a curved convex, wherein a peripheral portion of the second curved sheet is positioned on a peripheral portion of the first curved sheet, wherein a gap is defined between the concave surface and the convex surface, and wherein the electroactive pupil is positioned in the gap, and contacts the concave surface and the convex surface.

19. The pupil assembly of claim 17, wherein the electroactive pupil is substantially cylindrical or spherical shaped.

20. The pupil assembly of claim 17, wherein the electroactive pupil is made of an electroactive material.
